(19) United States
(12) Patent Application Publication
(10) Pub. No.: US 2012/0108643 A1
(43) Pub. Date: May 3, 2012

(54) METHOD OF INCREASING THE EXTENT OF ABSORPTION OF TIZANIDINE

(75) Inventors: Cara A. PELLEGRINI, Burlingame, CA (US); Paul Stark, Glasson Westmeath (IE)

(73) Assignee: King George Holdings IIA S.A.R.L.

(21) Appl. No.: 13/115,267

(22) Filed: May 25, 2011

Related U.S. Application Data
(63) Continuation of application No. 12/000,362, filed on Dec. 12, 2007, now abandoned.
Application No. 12/000,362 is a continuation of application No. 10/645,840, filed on Aug. 22, 2003, now abandoned.
Application No. 10/645,840 is a continuation-in-part of application No. 09/994,837, filed on Nov. 28, 2001, now Pat. No. 6,455,557.

(51) Int. Cl. A61K 31/433; A61P 25/14

(52) U.S. Cl. 514/362

(57) ABSTRACT

An article and method for increasing the extent of tizanidine absorption in a patient receiving tizanidine therapy. Tizanidine may be administered in the form of an immediate release tablet composition at or around the time food is consumed. The composition may be packaged in a container for distribution.

This application is a continuation-in-part of U.S. application Ser. No. 09/994,837, filed Nov. 28, 2001, the entire disclosure of which is incorporated herein by reference.

### FIELD OF THE INVENTION

This invention relates to a method and composition for improving the extent of absorption of tizanidine, and decreasing the occurrence of somnolence, in tizanidine drug therapy.

### BACKGROUND OF THE INVENTION

Tizanidine is pharmacologically characterized as a central-acting alpha₂ (α₂) adrenoceptor agonist that has myotonolytic activity useful in the treatment of spasticity in patients with cerebral or spinal injury, muscle spasm and pain. The imidazoline chemical structure of tizanidine is related to that of the anti-hypertensive drug clonidine and other alpha₂-adrenergic agonists; however, therapeutic indications are different between the two.

Tizanidine has one-tenth to one-fiftieth of the potency of clonidine in lowering blood pressure, while clonidine is ineffective in treating spastic conditions. This spectrum of activities is true of the 2-amino-imidazoline alpha₂ agonists in general, where differences in the ring structures to which the amino group attaches cause marked differences in pharmacologic properties. The article by Robert R. Ruffolo, Jr., titled “α-Adrenoreceptors: Molecular Biology, Biochemistry and Pharmacology” (Progress in Basic and Clinical Pharmacology series, Karger, 1991) provides general background on the alpha-adrenergic receptors. This article reviews the basis of alpha₁/alpha₂ subclassification, the molecular biology, signal transduction (G-protein interaction and location of the significant site for interaction and ligand-binding activity away from the 3′-terminus of alpha adrenergic receptors), agonist structure-activity relationships, receptor functions, and therapeutic applications for compounds exhibiting alpha-adrenergic receptor affinity. See also Chapleo, C. B., R. C. M. Butler, D. C. England, P. L. Myers, A. G. Roach, C. F. C. Smith, M. R. Stillings & I. F. Tulloch, “Heteroaromatic Analogues of the a₂-Adrenoreceptor Partial Agonist Clonidine,” Journal of Medicinal Chemistry, Vol. 32 (1989), pp. 1627-1630.

Anti-spastic efficacy has been demonstrated for tizanidine in placebo-controlled trials, with reduction in mean muscle tone scores of 21 to 37% versus 4 to 9% for patients receiving placebo. Maximum effects in some studies have been demonstrated to occur within two hours of administration. In one clinical study, tizanidine improved muscle tone in 60 to 82% of recipients, compared with 60 to 65% and 60 to 83% of recipients of baclofen and diazepam, respectively, two other anti-spastic agents. Tizanidine also reduced spasm frequency and clonus. Wagstaff A J; Bryson H M Drugs (NEW ZEALAND), Adis International Limited, Auckland, New Zealand, 53(3) pp. 435-452 (March 1997). The most common adverse effects associated with therapy are dry mouth and somnolence/drowsiness. Muscle strength, as assessed by objective means, does not appear to be adversely affected by tizanidine, and tizanidine recipients report subjective muscle weakness less often than recipients of baclofen or diazepam.

Tizanidine can be classified generically as an amino-imidazoline adrenergic agent. In chemical nomenclature, the molecule is described as 5-chloro-4-(2-imidazolin-2-ylamino)-2,1,3-benzothiadiazole and is identified with Chemical Abstracts Registry number 51322-75-9 and in Merck Index (Eleventh Addition, Merck & Co., 1989) at monograph no. 9409. The compound may form pharmaceutically acceptable acid addition salts, and is used as the hydrochloride salt in ZANAFLEX®, the commercially available prescription product for treatment of spasticity in the United States. Synthesis of the compound and its myotonolytic properties are disclosed in U.S. Pat. Nos. 3,843,668 and 4,053,617, the disclosures of which are hereby incorporated by reference.

Pharmacologic and electrophysiologic studies over the past 20 years have shown that tizanidine is a potent, centrally acting myotonolytic agent that principally affects spinal polysynaptic reflexes. This action arises from agonistic activity of the compound at noradrenergic alpha₂ receptors, resulting in both direct impairment of excitatory amino acid release from spinal interneurons and a concomitant inhibition of facilitatory coeruleospinal pathways. Similar alpha₂-receptor-mediated inhibition of interneuronal activity appears to underlie the additional antinociceptive and anticonvulsant activity of tizanidine reported in several species and test paradigms. Despite its structural and biochemical similarity to clonidine, the cardiovascular properties of tizanidine are mild and transitory in relation to its activity as a muscle relaxant. Smith H S; Barton A E, American Journal Of Hospice & Palliative Care (UNITED STATES), 17 (1) pp. 50-8 (January-February 2000).

### Pharmacokinetics

The absolute oral bioavailability of tizanidine is approximately 40% (CV=24%), due to extensive first-pass metabolism in the liver; approximately 95% of an administered dose is metabolized. Tizanidine is widely distributed throughout the body; mean steady state volume of distribution is 2.4 L/kg (CV=21%) following intravenous administration in healthy adult volunteers. Tizanidine is approximately 30% bound to plasma proteins, independent of concentration over the therapeutic range. ZANAFLEX® monograph, 2001 PHYSICIANS' DESK REFERENCE® Medical Economics Company, Inc. (publisher) Montvale, N.J. Tizanidine has linear pharmacokinetics over a dose of 1 to 20 mg.

In studies conducted on a tablet formulation, orally administered tizanidine was essentially completely absorbed and had a half-life of approximately 2.5 hours (coefficient of variation (CV)=33%). Id. Following administration of tizanidine, peak plasma concentrations occurred at 1.5 hours (CV=40%) after dosing. Earlier studies found that food increased Cₘₐₓ by approximately one-third and shortened time to peak concentration by approximately 40 minutes, but the extent of tizanidine absorption was not affected. Id.

However, the studies presented herein establish that those earlier conclusions relating to tizanidine absorption when an immediate release tablet form was administered with food were erroneous. Whereas the earlier studies concluded that food did not affect the extent of tizanidine absorption, the present studies show a significant increase in tizanidine absorption when administered with food. This discovery has led to the presently claimed invention involving methods involving increasing tizanidine bioavailability by administering the drug with food.

### BRIEF DESCRIPTION OF THE DRAWINGS

FIG. 1 is a plot of the plasma concentration of tizanidine in nanograms per milliliter versus the time elapsed from administration of the dosage form containing tizanidine. Four (4) plots are shown for tablet and capsule dosage forms administered with and without food.

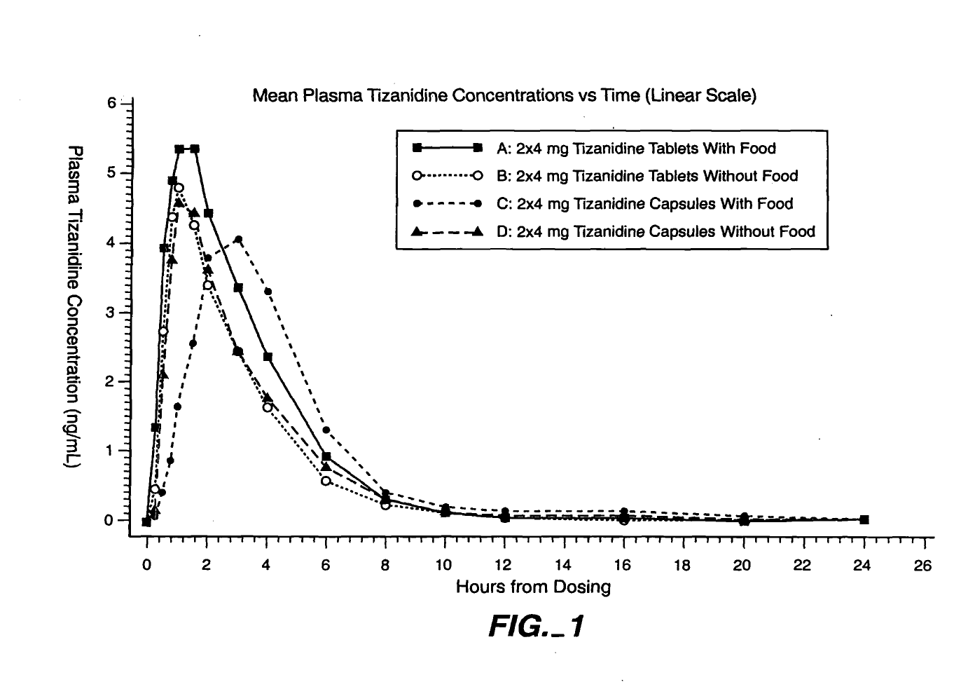

### SUMMARY OF THE INVENTION

It has surprisingly been found that administration of an immediate release tablet formulation of tizanidine taken at or around the time food is consumed results in an increase in the extent of absorption of tizanidine in patients receiving tizanidine therapy as compared with the tablet formulation administered without food. This result is different from results reported in earlier clinical studies of the tablet formulation that suggested that tizanidine, when taken with food, exhibited no effect on extent of absorption.

The present invention is also directed to methods of decreasing somnolence by taking an immediate release tizanidine tablet in a fasted state. These methods may involve providing information to patients receiving tizanidine therapy, prescribing physicians, pharmacists, or other health professionals, useful in modifying the pharmacokinetics of tizanidine in a patient receiving tizanidine therapy. This information may be used, for example, to decrease somnolence in patients receiving tizanidine therapy by providing information to administer an immediate release tablet formulation in a fasted state. The information may be provided by oral or written communication.

Preferably, the therapeutic amount is from about 0.5 mg to about 12 mg, and more preferably, from about 2 mg to about 8 mg, with the most preferred dosage being from about 2 mg to about 6 mg. Unit dosage forms are preferred.

Preferably, the food is a solid food with sufficient bulk and fat content that it is not rapidly dissolved and absorbed in the stomach. More preferably, the food is a meal, such as breakfast, lunch, or dinner. Advantageously, the dosage is administered to a patient from about 30 minutes prior, to about 2 hours after eating a meal, and most advantageously, the dosage is administered at substantially the same time as eating the meal. The terms “without food,” “fasted,” and “an empty stomach,” are defined to mean the condition of not having consumed solid food for about 1 hour before until about 2 hours after tizanidine administration.

Preferably, upon administering an immediate release tizanidine tablet formulation with food, the extent of tizanidine absorption, or AUCₗₐₛₜ, is increased by about 29%, as compared to administration of an immediate release tablet formulation in a fasted state. Preferably, upon administering an immediate release tizanidine tablet formulation without food, the maximum plasma concentration is decreased by at least about 20%, as compared to administration of an immediate release tablet with food.

With regard to the pharmacokinetic parameters set forth herein, Applicants note that it is well known that “bioequivalence” is defined by the Food and Drug Administration to be 80% to 125% of the referenced value, for example, of Cₘₐₓ or AUC. Thus, one of skill in the art would immediately recognize that a value 80% to 125% of the pharmacokinetic parameters set forth herein is bioequivalent to those pharmacokinetic parameters. Thus, the disclosure of the pharmacokinetic parameters herein immediately conveys to one or skill in the art a range of values that is bioequivalent.

Also, it is noted that the data for Cₘₐₓ and AUC in Table I is stated as the mean value, along with the standard error. It is noted that standard error, SE, is equal to the standard deviation, SD, of a sample, divided by the square root of the number in the sample. Thus, SD of the sample can be calculated by multiplying the SE by the square root of n.

For an 8-mg immediate release tizanidine tablet dose taken in a fasted state, the median Tₘₐₓ can range from about 0.8 to about 1.25 hours (which is 80%-125% of the median Tₘₐₓ), and can be about 1.0 hours. Mean Cₘₐₓ can range from about 3.19 to about 7.67 (mean Cₘₐₓ±SD), or from about 4.34 to about 6.79 (80%-125% of the mean Cₘₐₓ), or from about 5.18 to about 5.68 (mean Cₘₐₓ±SE), and can be about 5.43 ng/mL (mean Cₘₐₓ). The mean AUCₗₐₛₜ can range from about 6.93 to about 24.63 (mean AUCₗₐₛₜ±SD), or from about 12.62 to about 19.73 (80%-125% of the mean AUCₗₐₛₜ), or from about 14.79 to about 16.80 (mean AUCₗₐₛₜ±SE), and can be about 15.78 ng*hr/mL (mean AUCₗₐₛₜ). The mean AUC_{inf} can range from about 1.57 to about 37.51 (mean AUC_{inf}±SD), or from about 15.63 to about 24.43 (80%-125% of the mean AUC_{inf}), or from about 17.53 to about 21.55 (mean AUC_{inf}±SD), and can be about 19.54 ng*hr/mL (mean AUC_{inf}).

For an 8-mg immediate release tizanidine tablet dose taken in a fed state, the median Tₘₐₓ can range from about 1.12 to about 1.76 hours, and can be about 1.41 hours. The mean Cₘₐₓ can range from about 4.57 to about 9.03, or from about 5.44 to about 8.5, or from about 6.55 to about 7.05, and can be about 6.8 ng/mL. The mean AUCₗₐₛₜ can range from about 11.46 to about 29.61, or from about 16.24 to about 25.39, or from about 19.32 to about 21.30, and can be about 20.31 ng*hr/mL. The mean AUC_{inf} can range from about 5.88 to about 38.2, or from about 18.24 to about 28.5, or from about 20.26 to about 23.9, and can be about 22.08 ng*hr/mL.

For an 8-mg multiparticulate tizanidine formulation taken in a fasted state, the median Tₘₐₓ can range from about 0.81 to about 1.26 hours, and can be about 1.01 hours. The mean Cₘₐₓ can range from about 3.12 to about 7.6, or from about 4.29 to about 6.7, or from about 5.11 to about 5.61, and can be about 5.36 ng/mL. The mean AUCₗₐₛₜ can range from about 7.14 to about 24.84, or from about 12.79 to about 19.98, or from about 15.0 to about 16.98, and can be about 15.99 ng*hr/mL. The mean AUC_{inf} can range from about 14.41 to about 22.51, or from about 15.89 to about 20.13, and can be about 18.01 ng*hr/mL.

For an 8-mg multiparticulate tizanidine formulation taken in a fed state, the median Tₘₐₓ can range from about 2.4 to about 3.75 hours, and can be about 3.00 hours. The mean Cₘₐₓ can range from about 2.33 to about 6.81, or from about 3.66 to about 6.79, or from about 4.32 to about 4.82, and can be about 4.57 ng/mL. The mean AUCₗₐₛₜ can range from about 8.58 to about 26.28, or from about 12.62 to about 19.73, or from about 16.44 to about 18.42, and can be about 17.43 ng*hr/mL. The mean AUC_{inf} can range from about 16.66 to about 26.04, or from about 18.29 to about 23.37, and can be about 20.83 ng*hr/mL.

Another aspect of this invention is directed to providing information to patients receiving tizanidine therapy, prescribing physicians, pharmacists, or other health professionals, useful in modifying the pharmacokinetics of tizanidine in a patient receiving tizanidine therapy. This information may be used, for example, to increase the extent of tizanidine absorption from an immediate release tablet formulation. The information can be provided by oral or written communication.

In yet another aspect of this invention, an article of manufacture is provided comprising a container containing an immediate release tablet formulation comprising tizanidine or a pharmaceutically acceptable salt thereof. Preferably, the container holds the tizanidine (or its salt) composition in unit dosage form and is associated with printed labeling instructions advising of the increased extent of absorption when the tablet formulation is taken with food. The container may also include information relating to somnolence, and indicating that somnolence may be reduced by taking the immediate release tablet in a fasted state.

As used herein, an “immediate release” composition is one that allows all or substantially all of the tizanidine to be released from the dosage form in less than 60 minutes. Preferably, at least 75% of the tizanidine is released in less than 60 minutes. More preferably, at least 75% of the tizanidine is released in 30 minutes.

The term “multiparticulate” as used herein means a plurality of discrete particles, pellets, or mini-tablets, and mixtures or combinations thereof. If the oral form is a multiparticulate capsule, such hard or soft gelatin capsules can suitably be used to contain the multiparticulate. Alternatively, a sachet can suitably be used to contain the multiparticulate.

Preferably, the immediate release tablet pharmaceutical formulation is in the form of a conventional pressed tablet. The tablet formulation used in methods of the present invention can be prepared by any means. Preferably, the therapeutic amount of tizanidine is from about 0.5 mg to about 12 mg, more preferably from about 2 mg to about 8 mg, and most preferably from about 2 mg to about 6 mg.

The immediate release tizanidine tablets may include conventional excipients of the type used in pharmaceutical compositions. For example, the tablets may include pharmaceutically acceptable organic or inorganic carriers suitable for oral administration. Examples of such carriers include, but are not limited to, sugar spheres, diluents, hydrophilic polymers, lubricants, glidants (or anti-adherents), plasticizers, binders, disintegrants, surfactants, and pH modifiers.

Suitable diluents include, but are not limited to, microcrystalline cellulose, lactose, sucrose, fructose, glucose dextrose, and/or other sugars, dibasic calcium phosphate, calcium sulfate, cellulose, ethylcellulose, cellulose derivatives, kaolin, mannitol, lactitol, maltitol, xylitol, sorbitol, and/or other sugar alcohols, dry starch, dextrin, maltodextrin and/or other polysaccharides, inositol, and mixtures of any of the foregoing.

Suitable hydrophilic polymers include, but are not limited to, hydroxypropylmethyl cellulose, carbomers, polyethylene oxides, hydroxypropyl cellulose, hydroxyethyl cellulose, carboxymethylcellulose and its derivatives, sodium carboxymethylcellulose, carboxyvinylpolymers, polyvinyl alcohols, glucans, scleroglucans, mannans, xanthans, methylcellulose and, in general, cellulose, crosslinked polyvinylpyrrolidone, carboxymethyl starch, potassium methacrylate-divinylbenzene copolymer, hydroxypropylcyclodextrin, alpha-, beta-, gamma-cyclodextrin or derivatives, and other dextran derivatives, natural gums, seaweed extract, plant exudate, agar, agarose, algin, sodium alginate, potassium alginate, carrageenan, kappa-carrageenan, lambda-carrageenan, fucoidan, furcellaran, laminarin, hypnea, eucheuma, gum arabic, gum ghatti, gum karaya, gum tragacanth, guar gum, locust bean gum, quince psyllium, flax seed, okra gum, arabinogalactin, pectin, scleroglucan, dextran, amylose, amylopectin, dextrin, acacia, karaya, guar, a swellable mixture of agar and carboxymethyl cellulose, a swellable composition comprising methyl cellulose mixed with a sparingly cross-linked agar, a blend of sodium alginate and locust bean gum, and the like.

Suitable glidants (or anti-adherents) include, but are not limited to, colloidal silica, fumed silicon dioxide, silica hydrogel, talc, fumed silica, gypsum, kaolin, and glyceryl monostearate.

Suitable plasticizers include, but are not limited to, acetylated monoglycerides, butyl phthalyl butyl glycolate, dibutyl tartrate, diethyl phthalate, dimethyl phthalate, ethyl phthalyl ethyl glycolate, glycerin, propylene glycol, triacetin, citrate, tripropionin, diacetin, dibutyl phthalate, acetyl monoglyceride, polyethylene glycols, castor oil, triethyl citrate, polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisooctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate.

Suitable binders include, but are not limited to, starches, hydroxypropylmethyl cellulose, hydroxypropyl cellulose, ethyl cellulose, polyvinyl pyrrolidone, acacia, guar gum, hydroxyethylcellulose, agar, calcium carrageenan, sodium alginate, gelatin, saccharides (including, but not limited to, glucose, sucrose, dextrose and lactose), molasses, extract of Irish moss, panwar gum, ghatti gum, mucilage of isapol husk, carboxymethylcellulose, methylcellulose, veegum, larch arabolactan, polyethylene glycols, waxes, and mixtures of any of the foregoing.

Suitable disintegrants include, but are not limited to, starches, sodium starch glycollate, crospovidone, croscarmellose, microcrystalline cellulose, low substituted hydroxypropyl cellulose, pectins, potassium methacrylate-divinylbenzene copolymer, polyvinylalcohol, thylamide, sodium bicarbonate, sodium carbonate, starch derivatives, dextrin, beta cyclodextrin, dextrin derivatives, magnesium oxide, clays, bentonite, and mixtures of any of the foregoing.

Suitable surfactants include, but are not limited to, nonionic surfactants such as sorbitan sesquioleate, polyoxyethylene sorbitan monooleate, polyoxyethylene monostearate, glycerol monostearate, propylene glycol monolaurate, polyoxyethylene lauryl ether, polyoxyethylene cetyl ether, and polyoxyethylene hydrogenated castor oil, and ionic surfactants such as sodium dodecyl sulfate and benzalkonium chloride, and the like.

Suitable pH modifiers include, but are not limited to, organic acids such as citric acid, fumaric acid, tartaric acid, succinic acid, ascorbic acid, acetic acid, malic acid, glutaric acid and adipic acid, salts of any of the foregoing acids, salts of inorganic acids, and magnesium hydroxide.

Other aspects of the invention will become apparent upon a careful reading of this patent specification and claims.

### Assessment of Pharmacokinetic (PK) Studies and Somnolence by Cognitive Impairment Measurements:

Somnolence following tizanidine administration was studied by assessing the impact of somnolence on cognitive function as measured by Power of Attention (as defined herein) after each study treatment. Secondarily, orthostatic hypotension as a side effect was assessed by measuring decreases of at least 20 mm Hg in systolic or 10 mm Hg in diastolic blood pressure resulting from movement from the supine to the standing position.

The trials were conducted as a four-way crossover study, with a single dose for each treatment period, and with a minimum three-day washout period between study treatments. Up to ninety-six subjects were enrolled to ensure eighty subjects completed all four treatments. Healthy male or female volunteers, ages 18-55, within 20% of ideal body weight, were enrolled in the study. The volunteers had normal values for systolic and diastolic blood pressures, given their age and weight.

Eighty-eight subjects completed all four periods of the study. Pharmacokinetic parameters were calculated for all subjects who completed at least one treatment (N=96). Of the ninety-six subjects who participated in the study, eighty subjects were included in the summary statistics and statistical analysis of the pharmacokinetic parameters. The following groups of subjects were excluded from the statistical analysis of the pharmacokinetic parameters: subjects who vomited during an eight-hour period following a dose in one or more periods (N=3); subjects who did not complete all four periods (N=8); and subjects who were missing too many concentration data points within a period for one or more periods (N=5).

Multiparticulate, Dose, Duration, and Mode of Administration: The test product was tizanidine (“Treatment C” and “Treatment D”) 2×4 mg capsules, manufactured by Elan Pharmaceutical Technologies. “Treatment C” subjects received a single oral dose of two tizanidine 4 mg capsules, with food, taken with 240 mL of water. “Treatment D” subjects received a single oral dose of two tizanidine 4 mg capsules, fasted, taken with 240 mL of water.

Tablet, Dose, Duration, and Mode of Administration: The tablet product was ZANAFLEX® (tizanidine hydrochloride) (“Treatment A” and “Treatment B”) 2×4 mg tablets, manufactured by Brecon Pharmaceutical Limited. “Treatment A” subjects received a single oral dose of two ZANAFLEX® 4 mg tablets, with food, taken with 240 mL of water. “Treatment B” subjects received a single oral dose of two ZANAFLEX® 4 mg tablets, fasted, taken with 240 mL of water.

Cognitive Assessment: Simple Reaction Time, Digit Vigilance Task, Choice Reaction Time, Bond-Lader Visual Assessment Scale of Mood and Alertness were assessed at pre-dose, 0.75, 1.5, 2.5, and 6 hours post-dose. See the statistical methods section below for a discussion of the cognitive assessment system and its application.

Pharmacokinetic Evaluation: Plasma concentrations of tizanidine were determined following the single dose administration of each treatment (Treatment A: 2×4 mg immediate release tizanidine tablets with food, Treatment B: 2×4 mg immediate release tizanidine tablets without food, Treatment C: 2×4 mg tizanidine capsules with food, and Treatment D: 2×4 mg tizanidine capsules without food). The pharmacokinetic parameters Cₘₐₓ, Tₘₐₓ, AUC(0-t), AUC(0-inf), were calculated using noncompartmental methods.

Safety parameters assessed included medical history, physical examination, vital signs, orthostatic hypotension, 12-lead ECGs, clinical laboratory testing, adverse events, and cognitive assessment.

Statistical Methods:

Cognitive Assessment: The Cognitive Drug Research assessment system as used in this study has been described elsewhere in, e.g. Wesnes, K. A.; Garratt, C.; Wickens, M.; Gudgeon, A.; Oliver, S.; Effects of sibutramine alone and with alcohol on cognitive function in healthy volunteers, British Journal of Pharmacology, 49: 110-117 (2000) and Simpson, P. M.; Surmon, D. J.; Wesnes, K. A.; and Wilcock, G. R., The cognitive drug research computerized assessment system for demented patients: A validation study. International Journal of Geriatric Psychiatry 6: 95-102 (1991). Power of Attention was determined by the summation of scores from Simple Reaction Time, Digit Vigilance Task, and Choice Reaction Time. Degree of somnolence was derived from Self-rated Alertness, Self-rated Contentedness, and Self-rated Calmness formed from the Bond-Lader VAS scores. ANOVA model was used to detect treatment differences. The tests employed in conducting the assessment measure major aspects of cognitive function. The attentional tests are highly sensitive to fluctuations in the levels of alertness, which influence the ability to conduct everyday tasks such as driving.

Pharmacokinetic Evaluation: Plasma concentrations and pharmacokinetic parameters were summarized by treatment using summary statistics. A parametric, normal-theory general linear model was applied to the untransformed and log-transformed Cmax, AUC(0-t), and AUC(0-inf) parameter values. The primary comparisons were Treatment C versus Treatment A, Treatment D versus Treatment B, and Treatment C versus Treatment D. Comparisons of Tₘₐₓ were made using the Wilcoxon Signed Rank test.

Cognitive Assessment Results: The impairment in Power of Attention following capsules taken under fed conditions showed a delay of onset compared with the tables taken under fed conditions and capsules and tablets taken under fasted conditions. There was no impairment at 0.75 hours following capsules taken under fed conditions while the impairment was significant with the other three dosing groups. At 1.5 hours and 2.5 hours post dose, performance was impaired under all four dosing groups to a lesser extent than at 0.75 hours and the effect resolved completely by 6 hours post dose. The secondary measures showed the same pattern of change as Power of Attention.

Pharmacokinetic (PK) Results: Median and mean pharmacokinetic parameters following the four treatment regimens are shown in Table I.

TABLE I
Pharmacokinetic Data
Tizanidine Tablet/Capsule Study

| PARAMETER | Treatment A | Treatment B | Treatment C | Treatment D |
|---|---|---|---|---|
| Tₘₐₓ (hr) | 1.41 | 1.00 | 3.00 | 1.01 |
| Median value | | | | |
| Cₘₐₓ (ng/mL) | 6.80 | 5.43 | 4.57 | 5.36 |
| Mean value | [0.25] | [0.25] | [0.25] | [0.25] |
| AUCₗₐₛₜ | 20.31 | 15.78 | 17.43 | 15.99 |
| (ng * hr/mL) | [0.99] | [0.99] | [0.99] | [0.99] |
| Mean value | | | | |
| AUC_{inf} | 22.08 | 19.54 | 20.83 | 18.01 |
| (ng * hr/mL) | [1.82] | [2.01] | [2.54] | [2.12] |
| Mean value | | | | |

Treatment:
A = 2 × 4 mg Immediate Release Tizanidine Tablets with food
B = 2 × 4 mg Immediate Release Tizanidine Tablets without food
C = 2 × 4 mg Tizanidine Capsules with food
D = 2 × 4 mg Tizanidine Capsules without food
Values in brackets are the standard error

The values for Tmax were statistically compared between treatments and the median Tmax for the tablet fasted (1.0 hr) and capsule fasted (1.01 hr) were not different (p=0.0601). The median Tₘₐₓ for the capsule fed (3.0 hr) was significantly later than that for the tablet fed (1.41 hr) (p<0.0001) where p is Student's T value

Fasted administration of the capsule achieved a mean Cmax of 5.36 ng/mL at 1.01 hr compared to a mean Cmax of 5.43 ng/mL at 1.00 hr for the tablets administered in the fasted state. The AUC(0-t) of the capsule (15.99 ng*hr/mL) administered in the fasted state were similar. The tizanidine capsule met the established criteria for bioequivalence to the ZANAFLEX® tablet in the fasted state with respect to Cmax and AUC(0-t) in that there was no significant difference in either of the these parameters. The AUC(0-inf) values could not be calculated for a meaningful number of subjects in each treatment. The median Tmax values for the fasted capsule and tablet treatments were nearly identical (1.01 hr for the capsule and 1.0 hr for the tablet, p=0.077).

The pharmacokinetic study confirmed the bioequivalence, as defined by FDA guidelines, of the tizanidine capsule formulation and the ZANAFLEX® tablet in the fasted state. In the absence of a dose that would be within the range that would elicit the effects being studied, it is not appropriate to conclude that there is no absolute qualitative difference in effect between the tablet and capsules and the presence of food. It is clear, however, that the maximal plasma concentrations and extent of absorption in the presence of food are more marked for the tablet than the capsule.

The food effect on the PK parameters of tizanidine following the capsule administration was appreciable, as it resulted in an approximately 20% difference in Cₘₐₓ and AUC(0-t) compared to administration in the fasted state. The results for the ZANAFLEX® tablet, however, showed a more marked effect with Cmax approximately 23% higher and AUC(0-t) approximately 45% higher when administered with food. (Analyzed using log-transformed values).

Based on all safety data obtained during the study, administration of immediate release tizanidine tablets and capsules in fed and fasted conditions appeared to be safe and generally well tolerated by the healthy male and female subjects participating in the study. Asthenia and somnolence were the most common treatment-related side effects, following all treatments. Orthostatic hypotension was experienced during all treatment regimens by 24%-31% of subjects following dosing, with the majority of the episodes occurring between 1 and 2 hours post-dose. No treatment-related differences were noted for adverse events, clinical laboratory parameters, ECGs, or physical examinations.

The reformulation of tizanidine into a multiparticulate capsule resulted in a reduced food effect on Cmax and AUC compared to the commercial tablet formulation. The effect of food causing an increase in the Cmax and AUC of tizanidine was diminished by the new capsule formulation. The capsule formulation also resulted in a greater delay in absorption (median 2 hours) when administered with food compared to the tablet formulation (median 25 minutes). Administration of immediate release tizanidine tablets and capsules in fed and fasted conditions appeared to be safe and generally well tolerated by the healthy male and female subjects participating in the study.

The following non-limiting examples are provided by way of illustration, and are not intended to construe the scope of the invention.

Multiparticulate Pharmaceutical Compositions:

### Description of Individual Process Steps

| Stage | Description | Summary |
|---|---|---|
| 1 | Application Solution | Manufacture of application solution |
| | | containing active, anti adherent, binder |
| | | and purified water. |
| 2 | Immediate Release | Application of solution from Stage 1 onto |
| | Multiparticulates | inert sugar spheres (nonpareil beads) |
| | | to form drug loaded multiparticulates. |
| 3 | Encapsulation/Weight | Encapsulation of the multiparticulates |
| | sorting | from Stage 2 to achieve a unit dose |
| | | of the formulation. |
| | | Weight sorting of encapsulated product. |

For the manufacture of the Tizanidine HCl Application Solution, the relevant excipients were firstly weighed out. The binder (i.e., hydroxypropyl methylcellulose 3 cps) was mixed with purified water for at least 30 minutes or until completely dissolved. The active was then added and mixed for a further 15 minutes to form a solution. The anti adherent (i.e., silicon dioxide) was added finally, and mixed for a further 15 minutes.

For the manufacture of the Tizanidine HCl IR Multiparticulates, the Application Solution and the nonpareils (0.85-1.00 mm) were weighed. The nonpareils were pre-conditioned in the Glatt GPCG 30 (Glatt, Protech Ltd., Leicester, UK) fluid bed coating apparatus by placing the beads in the Glatt chamber, fluidised using air, and heated until the bypass temperature reached 55° C. The required amount of application solution was applied using the standard Glatt Wurster Process to achieve a target potency of 26.6 mg tizanidine per gram of multiparticulates. The target product temperature was 37° C. (Range 32-42° C.). An atomizing air pressure of 2.0 bar was used. The spray rate and inlet temperature were adjusted to achieve the required product temperature. The applied beads were dried for a further 10 minutes in the Glatt, allowed to cool for 5 minutes, and then discharged into HDPE containers. They were stored with desiccant.

For the manufacture of Tizanidine HCl IR Capsules, the required amount of IR Beads was filled into hard gelatin capsules. Size 3 capsules were used for the 2 mg and 4 mg strengths. Size 2 capsules were used for the 6 mg strength. The different capsule strengths were achieved by encapsulating the multiparticulates at different final fill weights. The filled capsules were then checkweighed.

### Example 1

### Immediate Release Multiparticulates

A Tizanidine HCl Application Solution was prepared as described in the Description of Individual Process Steps above according to the formulation in Table 1. The Tizanidine HCl Application Solution was then coated onto nonpareil seeds to a level of approximately 9.5% solids weight gain using a Glatt GPCG 30 (Glatt, Protech Ltd., Leicester, UK) fluid bed coating apparatus to form Immediate Release Multiparticulates as described in the Description of Individual Process Steps above.

TABLE 1
Tizanidine HCl Application Solution

| Ingredient | Amount (% w/w) |
|---|---|
| Tizanidine HCl (Novartis) | 3.59 |
| Hydroxypropyl Methylcellulose 3 cps | 4.96 |
| (Shin Etsu Chemical Co Ltd) | |
| Silicon Dioxide (USP grade, W.R.Grace | 1.65 |
| & Co.) | |
| Purified Water | 89.79 |

(b) Immediate Release Capsules

The Immediate Release Multiparticulates prepared according to Example 1(a) above were encapsulated into hard gelatin capsules (Capsugel Colmar, Colmar France) to the required dosage strength as described in the Description of Individual Process Steps above.

TABLE 2
Immediate Release Capsules

| | 2 mg Capsule | | 4 mg Capsule | | 6 mg Capsule | |
|---|---|---|---|---|---|---|
| | | % | | % | | % |
| Ingredient | mg/capsule | w/w | mg/capsule | w/w | mg/capsule | w/w |
| Tizanidine HCl | 2.29 mg | 3.0 | 4.58 mg | 3.0 | 6.87 mg | 3.0 |
| Hydroxypropyl | 3.16 mg | 4.2 | 6.32 mg | 4.2 | 9.48 mg | 4.2 |
| Methylcellulose | | | | | | |
| 3 cps | | | | | | |
| Silicon Dioxide | 1.05 mg | 1.4 | 2.10 mg | 1.4 | 3.15 mg | 1.4 |
| Nonpareil Beads | 68.7 mg | 91.4 | 137.4 mg | 91.4 | 206.1 mg | 91.4 |

For dissolution testing, USP type II (rotating paddles) dissolution apparatus was employed at 50 rpm using 500 ml 0.01M HCl maintained at 37±0.5° C. The final capsule had not less than 75% released after 0.5 hrs.

### Example 2

### (a) Immediate Release Multiparticulates

A Tizanidine HCl Application Solution is prepared as described in the Description of Individual Process Steps above according to the formulation in Table 3. The Tizanidine HCl Application Solution is then coated onto nonpareil seeds to a level of approximately 7.0% solids weight gain using for example a Glatt GPCG 5 (Glatt, Protech Ltd., Leicester, UK) fluid bed coating apparatus to form Immediate Release Multiparticulates as described in the Description of Individual Process Steps above.

TABLE 3
Tizanidine HCl Application Solution

| Ingredient | Amount (% w/w) |
|---|---|
| Tizanidine HCl | 3.59 |
| Hydroxypropyl Methylcellulose 6 cps | 2.50 |
| Silicon Dioxide | 1.65 |
| Purified Water | 92.26 |

Immediate Release Capsules

The Immediate Release Multiparticulates prepared according to Example 2(a) above are encapsulated into hard gelatin capsules to the required dosage strength as described in the Description of Individual Process Steps above.

TABLE 4
Immediate Release Capsules

| | 2 mg Capsule | | 4 mg Capsule | | 8 mg Capsule | |
|---|---|---|---|---|---|---|
| | | % | | % | | % |
| Ingredient | mg/capsule | w/w | mg/capsule | w/w | mg/capsule | w/w |
| Tizanidine HCl | 2.3 mg | 3.0 | 4.6 mg | 3.0 | 9.2 | 3.0 |
| Hydroxypropyl | 1.6 mg | 2.1 | 3.2 mg | 2.1 | 6.4 | 2.1 |
| Methylcellulose | | | | | | |
| 6 cps | | | | | | |
| Silicon Dioxide | 1.1 mg | 1.4 | 2.2 mg | 1.4 | 4.4 | 1.4 |
| Nonpareil Beads | 71.4 mg | 93.5 | 152.7 mg | 93.5 | 305.4 | 93.5 |

For dissolution testing, USP type II (rotating paddles) is employed at 50 RPM using 500 ml 0.01M HCl maintained at 37±0.5° C. The final capsule has not less than 75% released after 0.5 hrs.

### Example 3

### (a) Immediate Release Multiparticulates

A Tizanidine HCl Application Solution is prepared as described in the Description of Individual Process Steps above according to the formulation in Table 5. The Tizanidine HCl Application Solution is then coated onto nonpareil seeds to a level of approximately 9.5% solids weight gain using for example a Glatt GPCG 3 (Glatt, Protech Ltd., Leicester, UK) fluid bed coating apparatus to form Immediate Release Multiparticulates as described in the Description of Individual Process Steps above.

TABLE 5
Tizanidine HCl Application Solution

| Ingredient | Amount (% w/w) |
|---|---|
| Tizanidine HCl | 3.59 |
| Polyvinylpyrrolidone | 4.96 |
| Silicon Dioxide | 1.65 |
| Purified Water | 89.79 |

### (b) Immediate Release Capsules

The Immediate Release Multiparticulates prepared according to Example 3(a) above are encapsulated into hard gelatin capsules to the required dosage strength as described in the Description of Individual Process Steps above.

TABLE 6
Immediate Release Capsules

| | 2 mg Capsule | | 4 mg Capsule | | 6 mg Capsule | |
|---|---|---|---|---|---|---|
| | | % | | % | | % |
| Ingredient | mg/capsule | w/w | mg/capsule | w/w | mg/capsule | w/w |
| Tizanidine HCl | 2.29 mg | 3.0 | 4.58 mg | 3.0 | 6.87 mg | 3.0 |
| Polyvinyl- | 3.16 mg | 4.2 | 6.32 mg | 4.2 | 9.48 mg | 4.2 |
| pyrrolidone | | | | | | |
| Silicon Dioxide | 1.05 mg | 1.4 | 2.10 mg | 1.4 | 3.15 mg | 1.4 |
| Nonpareil Beads | 68.7 mg | 91.4 | 137.4 mg | 91.4 | 206.1 mg | 91.4 |

For dissolution testing, USP type II (rotating paddles) is employed at 50 RPM using 500 ml 0.01M HCl maintained at 37±0.5° C. The final capsule has not less than 75% released after 0.5 hrs.

### Example 4

### Immediate Release Multiparticulates

A Tizanidine HCl Application Solution is prepared as described in the Description of Individual Process Steps above according to the formulation in Table 7. The Tizanidine HCl Application Solution is then coated onto nonpareil seeds to a level of approximately 8.6% solids weight gain using for example a Glatt GPCG 30 (Glatt, Protech Ltd., Leicester, UK) fluid bed coating apparatus to form Immediate Release Multiparticulates as described in the Description of Individual Process Steps above.

TABLE 7
Tizanidine HCl Application Solution

| Ingredient | Amount (% w/w) |
|---|---|
| Tizanidine HCl | 2.54 |
| Hydroxypropyl Methylcellulose 3 cps | 3.95 |
| Talc | 1.50 |
| Purified Water | 91.56 |

(b) Immediate Release Capsules

The Immediate Release Multiparticulates prepared according to Example 4(a) above are encapsulated into hard gelatin capsules to the required dosage strength as described in the Description of Individual Process Steps above.

TABLE 8
Immediate Release Capsules

| | 4 mg Capsule | | 6 mg Capsule | | 12 mg Capsule | |
|---|---|---|---|---|---|---|
| | | % | | % | | % |
| Ingredient | mg/capsule | w/w | mg/capsule | w/w | mg/capsule | w/w |
| Tizanidine HCl | 4.56 | 2.5 | 6.84 | 2.5 | 13.68 | 2.5 |
| Hydroxypropyl | 7.09 | 3.9 | 10.64 | 3.9 | 21.28 | 3.9 |
| Methylcellulose | | | | | | |
| 3 cps | | | | | | |
| Talc | 2.73 | 1.5 | 4.10 | 1.5 | 8.2 | 1.5 |
| Nonpareil Beads | 167.44 | 92.1 | 251.16 | 92.1 | 502.32 | 92.1 |

For dissolution testing, USP type II (rotating paddles) is employed at 50 RPM using 500 ml 0.01 M HCl maintained at 37±0.5° C. The final capsule has not less than 75% released after 0.5 hrs.

### Example 5

### Immediate Release Multiparticulates

A Tizanidine HCl Application Solution is prepared as described in the Description of Individual Process Steps above according to the formulation in Table 9. The Tizanidine HCl Application Solution is then coated onto non-pareil seeds to a level of approximately 8% solids weight gain using for example a Glatt GPCG 30 (Glatt, Protech Ltd., Leicester, UK) fluid bed coating apparatus to form Immediate Release Multiparticulates as described in the Description of Individual Process Steps above.

TABLE 9
Tizanidine HCl Application Solution

| Ingredient | Amount (% w/w) |
|---|---|
| Tizanidine HCl | 7.00 |
| Hydroxypropyl Methylcellulose 3 cps | 4.00 |
| Fumaric acid | 7.00 |
| Talc | 2.50 |
| Purified Water | 79.50 |

(b) Immediate Release Capsules

The Immediate Release Multiparticulates prepared according to Example 5(a) above are encapsulated into hard gelatin capsules to the required dosage strength as described in the Description of Individual Process Steps above.

TABLE 10
Immediate Release Capsules

| | 2 mg Capsule | | 4 mg Capsule | | 6 mg Capsule | |
|---|---|---|---|---|---|---|
| | | % | | % | | % |
| Ingredient | mg/capsule | w/w | mg/capsule | w/w | mg/capsule | w/w |
| Tizanidine HCl | 2.5 | 2.5 | 5.0 | 2.5 | 7.5 | 2.5 |
| Hydroxypropyl | 1.4 | 1.4 | 2.8 | 1.4 | 4.2 | 1.4 |
| Methylcellulose | | | | | | |
| 3 cps | | | | | | |
| Talc | 1.2 | 1.2 | 2.4 | 1.2 | 3.6 | 1.2 |
| Fumaric acid | 2.5 | 2.5 | 5.0 | 2.5 | 7.5 | 2.5 |
| Nonpareil Beads | 90.6 | 92.4 | 181.2 | 92.4 | 271.8 | 92.4 |

For dissolution testing, USP type II (rotating paddles) is employed at 50 RPM using 500 ml 0.01 M HCl maintained at 37±0.5° C. The final capsule has not less than 75% released after 0.5 hrs.

### Example 7

### Immediate Release Tablet Formulation

A tizanidine mixture is prepared by mixing tizanidine with lactose, and the mixture is screened through a #30 mesh screen along with additional lactose. The mixture is then blended in a Hobart mixer, followed by screening through a #24 mesh screen.

A lactose pre-mix is prepared by mixing silicon dioxide with lactose. The mixture is blended in a Hobart mixer and then combined with lactose and screened through a #12 mesh screen.

A stearic acid pre-mix is prepared by passing stearic acid and lactose through a #12 mesh screen and mixing.

A portion of microcrystalline cellulose (MCC) is passed through a #28 mesh screen into a “V” blender and the tizanidine mixture is added. The remaining MCC, along with the lactose pre-mix and the remaining lactose is passed though a #28 mesh screen into the “V” blender and blended. The stearic acid pre-mix is passed through a #28 mesh screen and mixed in the “V” blender mix.

The blended mixture is compressed into 160 mg tablets (total mass), having a thickness of 2.5 mm, and a hardness of 5 to 8 Kp.

Article of Manufacture

The article of manufacture comprises a container holding an immediate release pharmaceutical composition suitable for oral administration of tizanidine in combination with printed labeling instructions providing a discussion of when a particular dosage form should be administered with food and when it should be taken on an empty stomach. The composition will be contained in any suitable container capable of holding and dispensing the dosage form and which will not significantly interact with the composition and will further be in physical relation with the appropriate labeling advising that an immediate release tablet dosage form exhibits an increased extent of absorption if taken with food, and exhibits less somnolence if taken on an empty stomach. The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

1. A method of using tizanidine in the treatment of spasticity comprising:
informing a patient in need of treatment that administration of a therapeutically effective amount of tizanidine in an immediate release pharmaceutical tablet formulation, with food, results in an increased extent of tizanidine absorption as compared to administration of an immediate release pharmaceutical tablet formulation without food;
wherein the patient administers the pharmaceutical tablet formulation with food.

2-39. (canceled)
